(19) United States
(12) Patent Application Publication
(10) Pub. No.: US 2024/0309357 A1
(43) Pub. Date: Sep. 19, 2024

(54) METHODS AND COMPOSITIONS FOR QUANTITATIVE, HIGH-THROUGHPUT PROTEIN BINDING MEASUREMENT

(71) Applicant: California Institute of Technology, Pasadena, CA (US)

(72) Inventors: Alexandre Lourenco, Pasadena, CA (US); Shichen Liu, Pasadena, CA (US); Matthew W. Thomson, Pasadena, CA (US)

(21) Appl. No.: 18/603,200

(22) Filed: Mar. 12, 2024

Related U.S. Application Data
(60) Provisional application No. 63/451,769, filed on Mar. 13, 2023.

(51) Int. Cl. C12N 15/10

(52) U.S. Cl. CPC C12N 15/1055; C12N 15/1065; C12N 15/1089

(57) ABSTRACT

Disclosed herein include methods, compositions, and kits suitable for use in determining the interaction between proteins and their targets. In some embodiments, the determination of the interaction between proteins and their targets is quantitative and in a high throughput manner.

### RELATED APPLICATIONS

This application claims the benefit under 35 U.S.C. § 119(e) of U.S. Provisional Patent Application Ser. No. 63/451,769, filed on Mar. 13, 2023, the content of this related application is incorporated herein by reference in its entirety for all purposes.

### BACKGROUND

### Field

The present disclosure relates generally to the field of molecular biology, especially methods and compositions for determining the interaction between proteins and their targets quantitively in a high throughput manner.

### Description of the Related Art

Recent developments in machine learning using large language models show promise uncovering sequence-structure and sequence-function relationships by analyzing massive amounts of natural protein sequence data. The insights obtained from these models are often too general to address specific protein-design needs. Although these insights on sequence-structure and sequence-function relationships can be refined with further data collection, it remains challenging to scale up the refining in an efficient manner. For example, current screening technologies, such as phage and yeast display, can screen binding affinities of variants of proteins. However, such technologies rank only a few variants of the screened pool using rough measurements like enrichment. Therefore, a high throughput platform with the capacity to measure quantitative binding properties of millions of protein variants in parallel over a short period of time is in need.

### SUMMARY

Disclosed herein include methods for determining binding between one or more proteins and a target. In some embodiments, the method comprises: providing a plurality of vectors each comprises a nucleic acid encoding a protein; contacting the plurality of vectors with a target in a condition allowing the proteins displayed on the surfaces of the vectors to specifically bind the target; separating the vectors that are bound to the target from the vectors that are not bound to the target; barcoding the nucleic acids from the vectors that are bound to the target to generate barcoded nucleic acids; analyzing the barcoded nucleic acids; and determining the binding between the proteins encoded by the plurality of vectors and the target. In some embodiments, each of the plurality of vectors displays the encoded protein on its surface and the encoded proteins from at least two of the plurality of vectors are different.

In some embodiments, determining the binding between the proteins encoded by the plurality of vectors and the target comprises determining the binding affinity between each of the encoded proteins and the target. In some embodiments, separating the vectors that are bound to the target from the vectors that are not bound to the target comprises removing the vectors displaying a protein not bound to the target.

In some embodiments, the barcoded nucleic acid from each of the vectors comprises the coding sequence of the protein and a barcode sequence selected from a diverse set of barcode sequences. In some embodiments, the barcode sequence comprises a randomer sequence. In some embodiments, the randomer sequence is a unique molecular identifier (UMI) sequence.

In some embodiments, analyzing the barcoded nucleic acids comprises obtaining sequence information of the barcoded nucleic acids. In some embodiments, analyzing the barcoded nucleic acids comprises determining abundance of the barcoded nucleic acids generated from the vectors that are bound to the target. In some embodiments, obtaining abundance of the barcoded nucleic acids comprises sequencing the barcoded nucleic acids and determining relative or absolute amount of sequence reads in sequences of the barcoded nucleic acids. In some embodiments, determining relative or absolute amount of sequence reads comprises determine the number of UMIs with different sequences associated with each coding sequence of the protein.

In some embodiments, the encoded proteins from at least five, ten, fifty, a hundred, a thousand, or ten thousand of the plurality of vectors are different. In some embodiments, at least one of the encoded proteins differ from one or more of the remaining encoded proteins by one amino acid, two amino acids, three amino acids, four amino acids, or five amino acids. In some embodiments, at least one of the encoded proteins differ from each of the remaining encoded proteins by one amino acid, two amino acids, three amino acids, four amino acids, or five amino acids.

In some embodiments, the target is, or comprises, a protein, a nucleic acid, an aptamer, a lipid, a polysaccharide, a cell, or a combination thereof. In some embodiments, the target is, or comprises, a receptor, a ligand, or antibody, or a fragment thereof. In some embodiments, the target is a cytokine receptor. In some embodiments, the target is IL-2Rβ, IL-2Rγ_{c}, IL-7Rα or TNFR 1.

In some embodiments, the target is immobilized or attached to a bead, or the target is partially or entirely embedded in a bead. In some embodiments, the target is attached to a bead. In some embodiments, the target is covalently conjugated to the bead. In some embodiments, the size of the bead is 1 nm-100 μm. In some embodiments, one or more additional targets are immobilized or attached to the bead, or one or more additional targets are embedded in the bead. In some embodiments, the targets attached to each bead are the same. In some embodiments, the targets attached to each bead are different. In some embodiments, the targets attached to all beads are the same. In some embodiments, the bead is a solid bead or a semi-solid bead. In some embodiments, the bead is a hydrogel bead, a polymer bead, or a magnetic bead.

In some embodiments, the one or more proteins comprises at least 100, 1000 or 10000 proteins. In some embodiments, the protein is a cytokine mimic. In some embodiments, the protein is an IL-2 mimic, an IL-7 mimic, or a TNF mimic. In some embodiments, the protein is at least 50% identical to a known cytokine in sequence. In some embodiments, the protein is at least 90% identical to a known cytokine in sequence. In some embodiments, the target is a cytokine receptor or a fragment thereof and the protein is at least 90% identical to a cytokine known to bind the receptor a fragment thereof in sequence. In some embodiments, the one or more proteins differ from a known protein by at least one amino acid, two amino acids, three amino acids, four amino acids, or five amino acids.

In some embodiments, the vector is a eukaryotic cell, a procaryotic cell, or a non-cell vector. In some embodiments, the vector is a liposome or an exosome. In some embodiments, the vector is a virus. In some embodiments, the vector is a phage. In some embodiments, the phage is selected from the group consisting of f1, fd, If1, Ike, Xf, Pf1, Pf3, λ, T2, T3, T4, T7, P2, P4, Phi X-174, MS2, Bacillus phage Phi29 and f2 phage. In some embodiments, the vector is a T7 phage. In some embodiments, the method comprises obtaining genomic nucleic acid from the phage. In some embodiments, the phage genomic nucleic acid comprises the nucleic acid encoding the protein. In some embodiments, the plurality of vectors comprise at least 100, 1000 or 10000 vectors. In some embodiments, the proteins displayed on one vector are the same. In some embodiments, the proteins displayed on one vector are different.

In some embodiments, contacting the plurality of vectors with the target comprises incubating the plurality of vectors and the target at 25° C.-45° C. In some embodiments, contacting the plurality of vectors with the target comprises incubating the plurality of vectors and the target for 5 minutes to 24 hours.

In some embodiments, barcoding the nucleic acids from the vectors that are bound to the target comprises barcoding the nucleic acids from the vectors that are bound to the target with a plurality of barcode oligonucleotides. In some embodiments, the barcode oligonucleotide comprises a barcode sequence and a barcoding primer. In some embodiments, the nucleic acid in each of the plurality of vectors comprises a barcoding primer binding region capable of binding to the barcoding primer. In some embodiments, the barcode sequence comprises a randomer sequence. In some embodiments, the randomer sequence is a unique molecular identifier (UMI) sequence. In some embodiments, the length of the UMI is at least 6 bp, 10 bp, 15 bp, 20 bp, or 25 bp. In some embodiments, the length of the UMI is 15 bp. In some embodiments, the sequence of the UMI in each barcode oligonucleotide of the plurality of barcode oligonucleotides is different from the sequences of the UMIs in any other barcode oligonucleotides of the plurality of barcode oligonucleotides. In some embodiments, the barcode oligonucleotide further comprises one or more PCR primer binding sites and/or one or more sequencing primer sequences. In some embodiments, the PCR primer binding site is a Read 1 sequence and/or the sequencing primer sequence is P5 and/or P7 sequence.

In some embodiments, barcoding the nucleic acids from the vectors that are bound to the target comprises hybridizing the barcoding primer with the barcoding primer binding region. In some embodiments, the barcoding primer binding region is about 1-300 bp upstream of the nucleic acid encoding a protein. In some embodiments, the length of the barcoding primer binding region is about 5-100 bp.

In some embodiments, barcoding the nucleic acids from the vectors that are bound to the target comprises generating a single-stranded DNA comprising the barcode sequence, using the barcoding primer and the nucleic acid encoding a protein as template. In some embodiments, the single-stranded DNA is generated through a single cycle of PCR.

In some embodiments, the method further comprises separating a portion of the plurality of vectors before contacting the plurality of vectors with the target. In some embodiments, the method comprises barcoding and analyzing the portion of the plurality of vectors to obtain a pre-binding abundance of the nucleic acids. In some embodiments, determining the binding between the proteins encoded by the plurality of vectors and the target comprises comparing the pre-binding abundance of the nucleic acids and an abundance of the barcoded nucleic acids.

In some embodiments, the method comprises obtaining the nucleic acid encoding a protein before barcoding.

In some embodiments, the method comprises amplifying the barcoded nucleic acids to generate amplified barcoded nucleic acids. In some embodiments, the amplification is through PCR.

In some embodiments, analyzing the barcoded nucleic acids comprises sequencing the amplified barcoded nucleic acids.

In some embodiments, the binding affinity is dissociation constant or apparent dissociation constant. In some embodiments, the dissociation constant between a protein of the one or more proteins and the target is at least 1 μM. In some embodiments, the dissociation constant between a protein of the one or more proteins and the target is at most 1 μM. In some embodiments, the dissociation constant between a protein of the one or more proteins and the target is 1 pM-1 μM.

In some embodiments, determining the binding between the proteins encoded by the plurality of vectors and the target comprises ranking the proteins by the binding affinity.

In some embodiments, the binding affinity is provided to a protein design model as an input.

Also disclosed herein include compositions and/or kits. The compositions and/or kits comprise a bead and a plurality of barcode oligonucleotides disclosed herein.

### BRIEF DESCRIPTION OF THE DRAWINGS

FIG. 1A-FIG. 1G depict non-limiting exemplary embodiments and data related to protein CREATE combining artificial intelligence with high-throughput experimentation for protein design. FIG. 1A depicts protein CREATE consisting of a generative artificial intelligence algorithm that proposed variants of protein sequences to be tested experimentally by a binding assay. The binding assay gathered data of high quality in a high throughput manner. The assay worked by using barcoded variants that can be counted using sequencing in a massively parallel fashion. Variant counts before and after the binding of a target of the variants allowed estimation of apparent dissociation constants (K_{d}^{app}). FIG. 1B depicts the workflow of the binding assay. In the binding assay, T7 phage nanoparticles displayed variants on their surface that were genetically encoded. Magnetic beads containing the desired binding partner (e.g., target) were added to the phage library and allowed to bind. Individual phage genomes were labeled with a primer containing a 15 bp randomized region that serves as a unique molecular identifier (UMI) to convert raw NGS reads into molecule counts for each variant during post-processing. FIG. 1C depicts the use of primers containing UMI to count two variants. Two variants displayed on phage (Neo2/15 and INF-γ) were counted by omitting reads containing duplicate sequences in the UMI region. Samples can be diluted following addition of the UMI to prevent saturation of the UMI region. FIG. 1D depicts result of the assay distinguishing between a known IL7R inhibitor and non-binders displayed phage. Two different dilutions of sample before and after bead binding are shown, explaining the difference in counted unique sequences. FIG. 1E-FIG. 1G depict the results after scaling up the assay to many more variants. A site saturation library of a known IL7R inhibitor was made, expressed on phage, and characterized using the experimental platform disclosed. FIG. 1E depicts the scaling of reads. Reads were scaled such that the total number of reads before and after binding to target on bead were equivalent and plotted. Poisson curves were plotted, given the assumption that the probability of observing a unique read for a variant follows a binomial distribution. FIG. 1F depicts ranking of 377 variants in the site saturation library. Enrichment factors for each variant were plotted by dividing the number of unique reads after binding to target by those observed before binding to target and then dividing by the enrichment score of the parent IL7R inhibitor. The enrichment score of the parent IL7R inhibitor was obtained by dividing the number of unique reads of the parent IL7R inhibitor after binding to target by that observed before binding. FIG. 1G depicts validation of dissociation constant. Surface-plasmon resonance (SPR) was used to characterize kinetic parameters for selected enriched and un-enriched variants identified by the assay using single-cycle kinetics.

FIG. 2 depicts non-limiting exemplary embodiments and data related to an overview of the experimental platform disclosed herein, with emphasis on the enabling features of using unique molecular identifiers

FIG. 3A-FIG. 3B depict non-limiting exemplary embodiments and data related to the results of the binding assay. FIG. 3A depicts the counts, binding prediction and dissociation constants of two variants. FIG. 3B depicts a comparison of dissociation constants measured using the platform disclosed herein to that measured using SPR.

FIG. 4A-FIG. 4B depict non-limiting exemplary embodiments and data related to schematic of Bayesian Inference Framework. FIG. 4A depicts that non-binding phage was used as a negative control to estimate the diluting effects of washing the beads after binding has on the global population of phage. This dilution value was used to estimate the amount of background expected for each variant, given the null hypothesis that it did not bind. FIG. 4B depicts stochastic simulations calculating the expected number of phages bound given a variant dissociation constant and initial population number. Bayesian inference was used to infer the most probable dissociation constant given the observed data. The simulations predicted dissociation constants for variants whose counts cannot be explained by dilution alone.

FIG. 5 depicts non-limiting exemplary embodiments and data related to comparison of dissociation constants measured using the quantitative, high-throughput phage display platform to the dissociation constants measured using SPR with data collected by Silva et al. (2019).

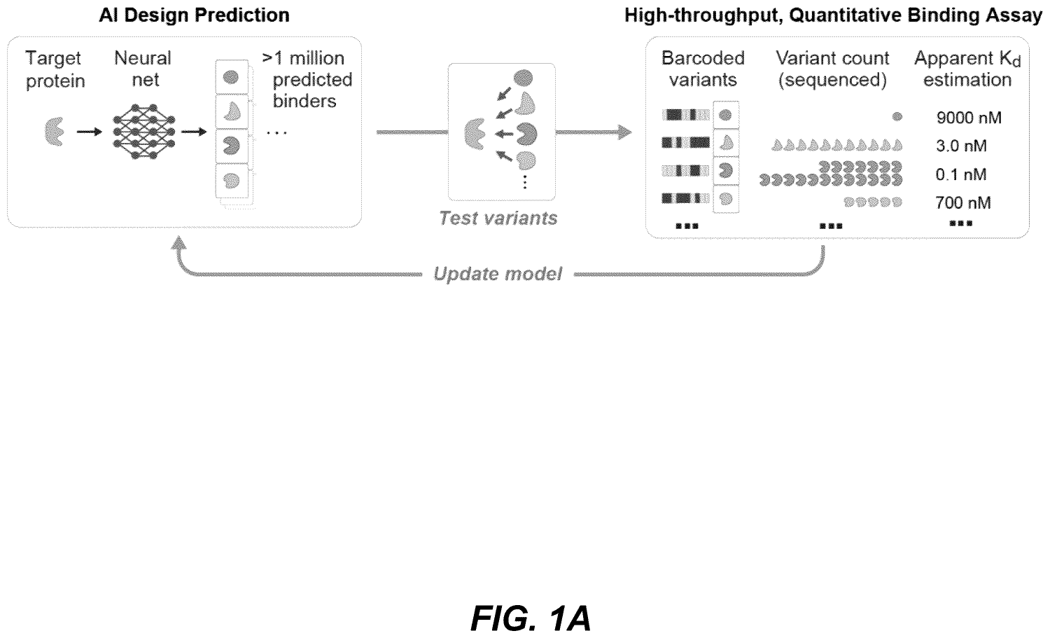

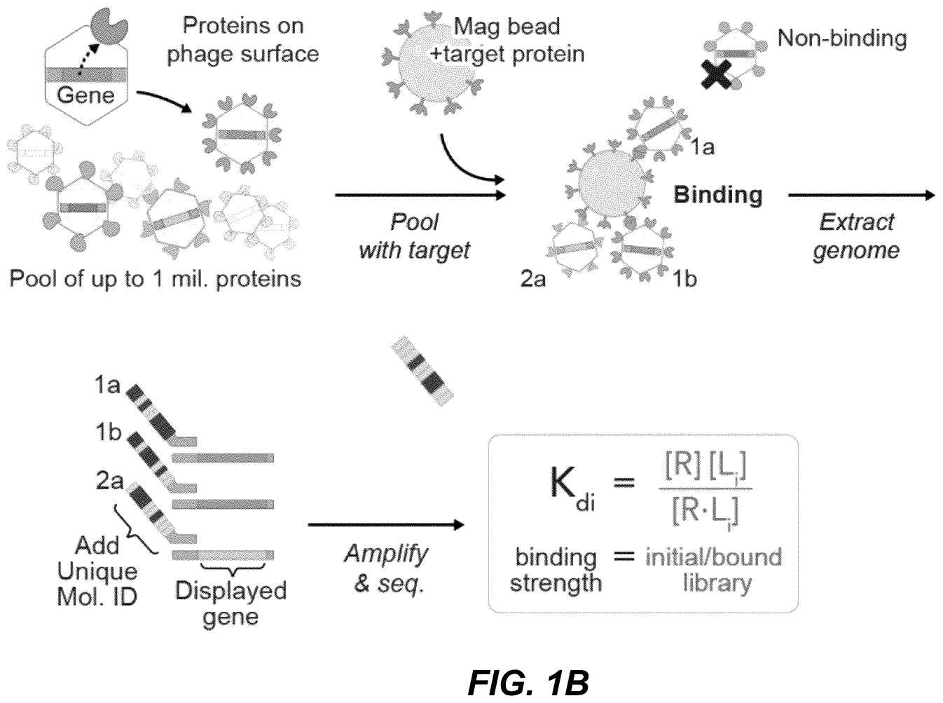

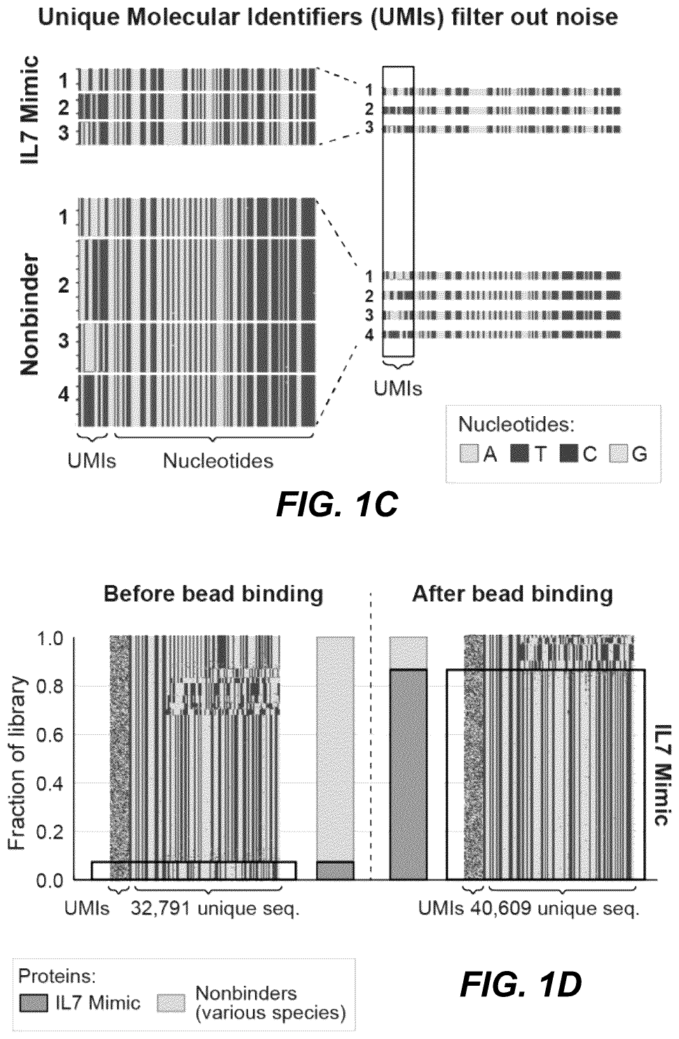

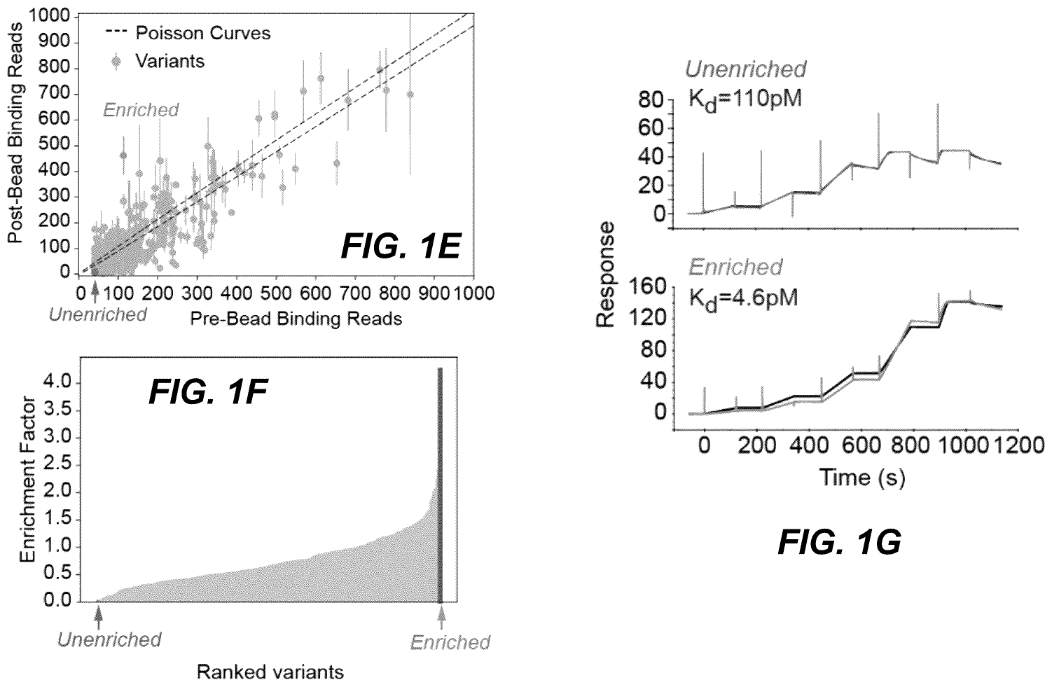

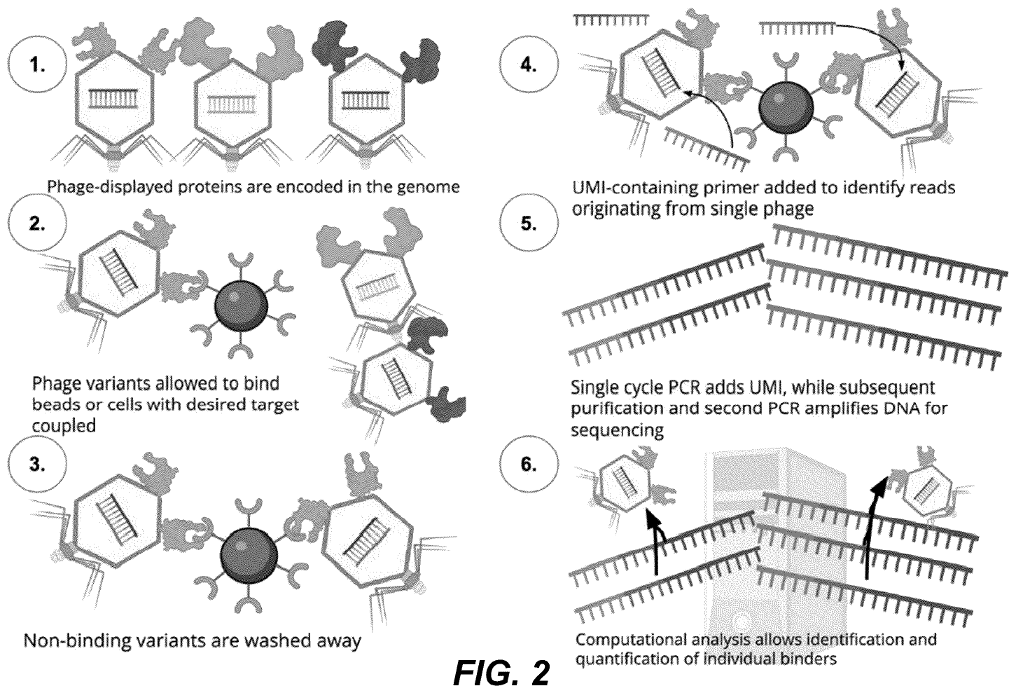

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “affinity,” “binding affinity” or “binding strength” are used interchangeably. They refer to the strength of the sum total of non-covalent interactions between a binding site of a molecule and its binding partner.

As used herein, the term “association constant” refers to measures of the strength of interaction between a compound (e.g., protein) and a target. Association constant is the reciprocal of dissociation constant and is calculated as:

$$association constant = \frac{[ compound \cdot target ]}{[ compound ] [ target ]} ,$$

in which [compound·target] is the molar concentration of complex formed between the compound (e.g., protein) and the target and [compound] and [target] are the molar concentration of the compound (e.g., protein) and the target that are not in the form of complex, respectively.

As used herein, the term “dissociation constant” or “K_{d}” refers to a measure of strength of interaction between a compound and a target. Dissociation constant is the reciprocal of association constant.

As used herein, the term “avidity” refers to a measure of the overall strength of the complex formed between a compound (e.g., protein) and a target. It is dependent on three major parameters: affinity and valency of the compound (e.g., protein) and the target, and structural arrangement of the parts that interact.

As used herein, the term “dissociation rate” refers to the rate or speed, at which a compound dissociates from a target.

As used herein, the term “association rate” refers to the rate or speed, at which a compound associates with a target.

As used herein, the term “enrichment factor” refers to the degree of enrichment of a given variant in the binding assay, compared to the parent protein. The enrichment factor of a variant if calculated by dividing the enrichment score of the variant by the enrichment score of the parent protein. The enrichment score is calculated by dividing the number of unique reads post-binding for a given variant or the parent protein by the number of unique reads pre-binding of that given variant or the parent protein. Thus, the enrichment factor of the parent sequence, for example, is by definition 1.

As used herein, the term “nucleic acids” includes any and all forms of alternative nucleic acid containing modified bases, sugars, and backbones including peptide nucleic acids and aptamers, optionally, with stem loop structures.

As used herein, the term “polypeptide” is used interchangeably with “peptide” and “protein.” They refer to a sequence of subunit natural amino acids, amino acid analogs including unnatural amino acids. Peptides include polymers of amino acids having the formula H₂NCHRCOOH and/or analog amino acids having the formula HRNCH₂COOH. The subunits are linked by peptide bonds (i.e., amide bonds), except as noted. Often all subunits are connected by peptide bonds. The polypeptides can be naturally occurring, processed forms of naturally occurring polypeptides (such as by enzymatic digestion), chemically synthesized or recombinantly expressed. Preferably, the polypeptides are chemically synthesized using standard techniques. The polypeptides can comprise D-amino acids (which are resistant to L-amino acid-specific proteases), a combination of D-and L-amino acids, β amino acids, and various other “designer” amino acids (e.g., (β-methyl amino acids, Cα-methyl amino acids, and Nα-methyl amino acids) to convey special properties. Synthetic amino acids include ornithine for lysine, and norleucine for leucine or isoleucine. Hundreds of different amino acid analogs are commercially available. In general, unnatural amino acids have the same basic chemical structure as a naturally occurring amino acid, i.e., a carbon that is bound to a hydrogen, a carboxyl group, an amino group, and an R group. In addition, polypeptides can have non-peptide bonds, such as N-methylated bonds (—N(CH₃)—CO—), ester bonds (—C(R)H—C—O—O—C(R)—N—), ketomethylen bonds (—CO—CH₂—), aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH—CH—), retro amide bonds (—NH—CO—), peptide derivatives (—N(R)—CH₂—CO—), wherein R is the “normal” side chain. These modifications can occur at any of the bonds along the peptide chain and even at several (e.g., 2-3) at the same time. For example, a peptide can include an ester bond. A polypeptide can also incorporate a reduced peptide bond, i.e., R₁—CH₂—NH—R₂, where R₁ and R₂ are amino acid residues or sequences. A reduced peptide bond can be introduced as a dipeptide subunit. Such a polypeptide would be resistant to protease activity, and would possess an extended half-live in vivo. The compounds can also be peptoids (e.g., N-substituted glycines), in which the sidechains are appended to nitrogen atoms along the molecule's backbone, rather than to the a-carbons, as in amino acids.

As used herein, the term “polysaccharide” means any polymer (homopolymer or heteropolymer) made of subunit monosaccharides, oligimers or modified monosaccharides. The linkages between sugars can include acetal linkages (e.g., glycosidic bonds), ester linkages (e.g., phophodiester linkages), amide linkages, and ether linkages.

As used herein, the term “cytokine” as used herein refers to cell signaling molecules that regulate the immune system's response to inflammation and infection and aid cell to cell communication in immune responses. Examples include chemokines, interferons, interleukins (e.g., IL-2 or IL-7), lymphokines, and tumor necrosis factors (TNF-α).

As used herein, the term “IL-2 receptor common gamma” or “IL-2Rγ_{c}” or “ IL-2RG” refers to the IL-2 gamma receptor and is a member of the type I cytokine receptor family that is a cytokine receptor subunit to the receptor complexes for at least six different interleukin receptors including, but not limited to, IL-2, IL-4, IL-7, IL-9, IL-15, and IL-21 receptors. The “hIL-2 receptor common gamma” or “hIL-2Rγ_{c}” or “hIL-2RG” refers to the human IL-2 gamma receptor.

As used herein, the term “IL-2Rβ receptor” or “IL-2R receptor beta” or “IL-2RB receptor” refers to the IL-2 beta receptor. “hIL-2Rβ receptor” or “hIL-2R receptor beta” or “hIL-2RB receptor” refers to the human IL-2 beta receptor.

As used herein, the term “IL2RBG” or “IL-2Rβγ_{c}” refers to the IL-2Rβ and IL-2Rγ_{c} heterodimer. “hIL2RBG” or “hIL-2Rβγ_{c}” refers to the hIL-2Rβ and hIL-2Rγ_{c} heterodimer. IL-2Rβγ_{c} is also known as the intermediate affinity IL-2 Receptor.

As used herein, the term “IL-2 antagonist” or “IL-2R antagonist” refers to a compound that opposes one or more actions of IL-2 or one or more activities of IL-2. The term antagonist refers to both full antagonists and partial antagonists. For example, a “partial antagonist” is an antagonist that does not-fully interrupt the biochemical effect of IL-2, but that is sufficient to interrupt selected targeted cellular and/or physiological activities promoted by IL-2. An antagonist of IL-2 might, under certain biological scenarios, have ability to induce IL-2-like signaling on its own (i.e., pSTAT5 signaling). In some embodiments, the ability to induce IL-2-like signaling is at a lower level than the signaling induced by IL-2.

Proteins have proven to be useful agents in a variety of fields from serving as blockbuster drugs to complex catalysis for chemical manufacture. However, rational engineering of proteins remains difficult. Rapid progress in the development of protein large language models (LLMs) has made it possible to efficiently search sequences of functional proteins distally related to natural ones. However, methods of rapidly testing these generated sequences and appropriately updating the generative models for generating these protein sequences remains a core challenge.

Various display technologies, such as phage, yeast, or ribosome display, coupled with next-generation sequencing (NGS) can be used to screen for desirable sequences. However, these technologies are inherently qualitative, because the results (e.g., enrichment scores) derived from these assays are not tied to biochemical parameters, suffer from false positives, and can require multiple rounds of biopanning, further complicating the use of the data in training generative models.

As a result, experimental characterization of LLM-derived protein candidates is rare. Moreover, the scope and scale of the experimental characterization are usually limiting, due to requirement to screen and sequence individual variants. Characterization of LLM-derived protein candidates has been limited to the expression and purification of ˜100 variants from an in vitro expression system. This scale of available in vitro expression system can facilitate the testing of the generated sequences, but cannot meet the requirement for updating the generative models. Moreover, these large language models are trained on natural sequences. Therefore, they often do not perform satisfyingly when generating unexplored regions of sequences. Overcoming this issue would also require updating the models with a large number of artificially generated sequences that are characterized. Relieving this design bottleneck could enable large-scale iterative and functional testing of proteins with a greater search space than that explored by evolution.

Disclosed herein include a Protein CREATE (computational redesign via an experiment-augmented training engine), an integrated computational and experimental pipeline that incorporate an actor-critic reinforcement learning framework to update protein large language models with quantitative binding and functional data collected from experimental workflows leveraging next generation sequencing and phage display with single-molecule readouts. Protein CREATE disclosed herein was used to assay binders to IL-2 receptor beta and gamma chains, IL-7 receptor alpha, and TNF receptor-1. These results were fed back into Protein CREATE to refine the initial generative models and demonstrated the feasibility of closed-loop design cycles.

In some embodiments, the Protein CREATE updated protein large language models with high-throughput, quantitative data derived from binding assays. In some embodiments, bacteriophages were used as genetically encoded nanoparticles to display protein variants of interest, where they can bind to a bait/target attached to a magnetic bead. A primer containing unique molecular identifier (UMI) hybridized to the phage genome and permitted identification of individual phage counts from raw sequencing read data. A Bayesian inference pipeline then converted those molecule counts into dissociation constant (K_{d}) estimates for each protein variant displayed by a phage, which were used to update the protein large language model for subsequent design-build-test cycles. In some embodiments, a site-saturation library of a known IL-7Ra binders were characterized, demonstrating the sensitivity of the binding assay. Both tighter and weaker binders were discovered and further validated using surface plasmon resonance (SPR). Despite the relative strength of all variants tested, the binding assay workflow disclosed herein was able to distinguish between variants with single-picomolar differences in dissociation constant, establishing the utility of the assay for generating high-quality training data for the generative model.

Disclosed herein include methods for determining binding between one or more proteins and a target. In some embodiments, the method comprises: providing a plurality of vectors each comprises a nucleic acid encoding a protein; contacting the plurality of vectors with a target in a condition allowing the proteins displayed on the surfaces of the vectors to specifically bind the target; separating the vectors that are bound to the target from the vectors that are not bound to the target; barcoding the nucleic acids from the vectors that are bound to the target to generate barcoded nucleic acids; analyzing the barcoded nucleic acids; and determining the binding between the proteins encoded by the plurality of vectors and the target. In some embodiments, each of the plurality of vectors displays the encoded protein on its surface and the encoded proteins from at least two of the plurality of vectors are different.

### Specific Binding and Binding Affinity

Binding between a compound (e.g., protein) to a target can be either specific or non-specific. Specific binding is detectably higher in magnitude and distinguishable from non-specific binding. Specific binding can be the result of formation of bonds between particular functional groups or particular spatial fit (e.g., lock and key type) whereas nonspecific binding is usually the result of van der Waals forces. In some embodiments, the binding assayed using the methods disclosed herein is specific binding.

A compound can often bind to more than one target specifically. Similarly, variants of a compound can bind to the same target specifically, but of different strengths. In some embodiments, variants of a compound bind to a target non-specifically. Different degrees of specific binding can be distinguished from one another as can specific binding from nonspecific binding. Specific binding often involves an apparent association constant of 10³ M⁻¹ or higher. Specific binding can additionally or alternatively be defined as a binding strength more than three standard deviations greater than background represented by the mean binding strength of empty control (i.e., having no compound, where any binding is nonspecific binding to the support).

An apparent association constant includes avidity effects or cooperative binding if present. Specifically, if a target shows multivalent binding to multiple molecules of the same compound the apparent association constant is a value reflecting the aggregate binding of the multiple molecules of the same compound to the target. The theoretical maximum of the avidity is the product of the multiple individual association constants, but in practice the avidity is usually a value between the association constant of individual bonds and the theoretical maximum. In some embodiments, different compounds in an assay have different degrees of binding strength to the target and some compounds can bind with higher or lower apparent association constants than other compounds.

Degrees of binding can be reflected by binding strength/affinity. In some embodiments, determining the binding between the proteins displayed by the plurality of vectors and the target comprises determining the binding affinity between each of the encoded proteins and the target. Binding strength can be measured by relative or absolute amount of sequence reads in sequences of barcoded nucleic acids, enrichment factor, enrichment score, association constant, dissociation constant, dissociation rate, or association rate, or a composite measure of stickiness which may include one or more of these measures. If a term used to define binding strength is referred to as “apparent” what is meant is a measured value without regard to multivalent bonding. For example, the measured value of an association constant under conditions of multivalent bonding includes a plurality of effects due to monovalent bonding among other factors. Unless otherwise specified, binding strength can refer to any of these measures referred to above. In some embodiments, the binding strength measured using methods disclosed herein is indicated by dissociation constant.

In some embodiments, the enrichment factor of a compound is 0-10 (e.g., 0.01, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 3, 4, 5, 6, 7, 8, 9 or 10 or a number between any two of these values). In some embodiments, the dissociation constant of a compound is less than 10 μM. In some embodiments, the dissociation constant of a compound is less than 1 μM (e.g., 0.01 nM, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2 nM, 2.1 nM, 2.2 nM, 2.3 nM, 2.4 nM, 2.5 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, or a number between any two of these values). In some embodiments, the apparent dissociation constant of a compound is less than 1 μM (e.g., 0.01 nM, 0.1 nM, 0.2 nM, 0.3 nM, 0.4 nM, 0.5 nM, 0.6 nM, 0.7 nM, 0.8 nM, 0.9 nM, 1 nM, 1.1 nM, 1.2 nM, 1.3 nM, 1.4 nM, 1.5 nM, 1.6 nM, 1.7 nM, 1.8 nM, 1.9 nM, 2 nM, 2.1 nM, 2.2 nM, 2.3 nM, 2.4 nM, 2.5 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, or a number between any two of these values).

### Compounds and Targets

Many different classes of compounds or combinations of classes of compounds can be assayed using the methods disclosed herein. Classes of compounds include nucleic acids and their analogs, polypeptides, polysaccharides, organic compounds, inorganic compounds, polymers, lipids, and combinations thereof. Many types of compounds can be synthesized in a step-by-step fashion. Such compounds include polypeptides, beta-turn mimetics, polysaccharides, phospholipids, holitiones, prostaglandins, steroids, aromatic compounds, heterocyclic compounds, benzodiazepines, oligomeric N-substituted glycines and oligocarbamates. Compounds can be constructed by the encoded synthetic libraries (ESL) method described in WO 1995/012608, WO 1993/006121, WO 1994/008051, WO 1995/035503 and WO 1995/030642, which are incorporated by reference by their entirety.

In some embodiments, the compounds are proteins (e.g., cytokines and/or cytokine mimics). The proteins can also be generated by phage display methods. The protein can have a size of 30-1000 residues (e.g., 30, 40, 50, 60, 70, 80, 90, 100, 200, 300, 400, 500, 600, 700, 800, 900, 1000 residues or number between any two of these values). The compounds tested can be natural or synthetic. The compounds can comprise linear or branched heteropolymeric compounds based on any of a number of linkages or combinations of linkages (e.g., amide, ester, ether, thiol, radical additions, and metal coordination), dendritic structures, circular structures, cavity structures or other structures with multiple nearby sites of attachment that serve as scaffolds upon which specific additions are made. The compounds can be naturally occurring or non-naturally occurring.

Many different classes of targets can be used in the methods disclosed herein. Classes of targets include nucleic acids and their analogs, polypeptides, polysaccharides, organic compounds, inorganic compounds, polymers, lipids, cells, and combinations thereof. In some embodiments, the target is, or comprises, a protein, a nucleic acid, an aptamer, a lipid, a polysaccharide, a cell, or a combination thereof. For example, the target can be, or can comprise, a receptor, a ligand, or antibody, or a fragment thereof. Many types of targets can be synthesized in a step-by-step fashion. Such targets include polypeptides, beta-turn mimetics, polysaccharides, phospholipids, holitiones, prostaglandins, steroids, aromatic compounds, heterocyclic compounds, benzodiazepines, oligomeric N-substituted glycines and oligocarbamates. In some embodiments, the targets are also proteins (e.g., cytokine receptor or antibody). The targets used herein can be natural or synthetic. The targets can comprise or consist of linear or branched heteropolymeric compounds based on any of a number of linkages or combinations of linkages (e.g., amide, ester, ether, thiol, radical additions, and metal coordination), dendritic structures, circular structures, cavity structures or other structures with multiple nearby sites of attachment that serve as scaffolds upon which specific additions are made. The targets can be naturally occurring or non-naturally occurring.

In some embodiments, the compounds are cytokines and/or cytokine mimics, while the targets are receptors (e.g., cytokine receptor). Many cell functions are regulated by members of the cytokine receptor superfamily. Signaling by these receptors depends upon their association with Janus kinases (JAKs), which couple ligand binding to tyrosine phosphorylation of signaling proteins recruited to the receptor complex. Among these are the signal transducers and activators of transcription (STATs), a family of transcription factors that contribute to the diversity of cytokine responses.

The cytokine receptor can be a type I cytokine receptor. Type I cytokine receptors share a common amino acid motif (WSXWS) in the extracellular portion adjacent to the cell membrane. The cytokine receptor can be a type II cytokine receptor. Type II cytokine receptors include those that bind type I and type II interferons, and those that bind members of the interleukin-10 family (e.g., interleukin-10, interleukin-20 and interleukin-22).

Type I cytokine receptors can include: (i) interleukin receptors, such as the receptors for IL-2, IL-3, IL-4, IL-5, IL-6, IL-7, IL-9, IL-11, IL-12, IL13, IL-15, IL-21, IL-23 and IL-27; (ii) colony stimulating factor receptors, such as the receptors for erythropoietin, GM-CSF, and G-CSF; and (iii) hormone receptor/neuropeptide receptor, such as hormone receptor and prolactin receptor. Members of the type I cytokine receptor family comprise different chains, some of which are involved in ligand/cytokine interaction and others are involved in signal transduction. For example, the IL-2 receptor comprises an α-chain, a β-chain and a γ-chain.

Interleukin 2 (IL-2) is a Type I cytokine receptor. IL-2 binds to the IL-2 receptor (IL-2R), which has three forms, generated by different combinations of three different proteins, often referred to as “chains” or “subunits”: α, β and γ. These subunits are also parts of receptors for other cytokines. For example, the IL-2 receptor common gamma chain (also known as CD132) is shared between the IL-2 receptor, IL-4 receptor, IL-7 receptor, IL-9 receptor, IL-13 receptor and IL-15 receptor. The β and γ chains of the IL-2R are members of the type I cytokine receptor family. The three receptor chains are expressed separately and differently on various cell types and can assemble in different combinations and orders to generate low, intermediate, and high affinity IL-2 receptors. The a chain binds IL-2 with low affinity; the combination of β and γ together form a complex that binds IL-2 with intermediate affinity, primarily on memory T cells and NK cells; and all three receptor chains form a complex that binds IL-2 with high affinity (K_{d} of about 10⁻¹¹ M) on activated T cells and regulatory T cells.

IL-2 promotes the differentiation of T cells and, therefore, plays a key role in long-term cell-mediated immunity. Thus, searching for IL-2 mimics has been a focus of research on immunotherapies. A de novo protein immunotherapeutic, Neoleukin-2/15 (also known as Neo-2/15), has recently been described. Neo-2/15 is a 100-residue de novo protein that mimics the function of both human interleukin-2 (hIL-2) and human interleukin-15 (hIL-15). Similar to IL-2, Neo-2/15 also binds to heterotrimeric receptor IL-2Rβγ_{c} with greater affinity. To accomplish its biological function, Neo-2/15 induces the hetero-dimerization of two IL-2 cell membrane receptors, the IL-2 receptor beta (IL-2Rβ) and the IL-2 receptor common gamma IL-2Rγ_{c}.

Interleukin-7 (IL-7) is an immunostimulatory cytokine member of the IL-2 superfamily and plays an important role in an adaptive immune system by promoting immune responses. This cytokine activates immune functions through the survival and differentiation of T cells and B cells, survival of lymphoid cells, stimulation of activity of natural killer (NK) cell. IL-7 also regulates the development of lymph nodes through lymphoid tissue inducer (LTi) cells and promotes the survival and division of naive T cells or memory T cells. Furthermore, IL-7 enhances immune response in human by promoting the secretion of IL-2 and Interferon-γ (INF-γ). The IL-7 receptor is made up of two chains: the IL-7 receptor-α chain (IL-7Rα or CD127) and common-y chain receptor (CD132). The common-γ chain receptor is shared with various cytokines. IL-7 receptor is expressed on various cell types, including naive and memory T cells.

Tumor necrosis factor-a (TNF or TNF-a) is a multifunctional cytokine mediating pleiotropic biological functions in both health and disease states. TNF is secreted primarily by monocytes and macrophages, but can also be secreted by other cell types. The list of processes regulated by TNF is extensive, and includes inflammation, immunoregulation, cytotoxicity and antiviral effects. The numerous biological effects of TNF are mediated by two transmembrane receptors, the 55 kDa Type I receptor (TNFR1 or CD120a) and the 75 kDa Type II receptor (TNFR2 or CD120b). Although both TNFR1 and TNFR2 demonstrate strong affinity for TNF-α, these two receptors demonstrate no apparent homology in their cytoplasmic (i.e., intracellular) domains.

In some embodiments, the compound to be tested using the method disclosed herein is a cytokine or cytokine mimic. In some embodiment, the cytokine is an interferon, interleukin-10, interleukin-20, interleukin-22, IL-2, IL-3, IL-4, IL-5, IL-6, IL-7, IL-9, IL-11, IL-12, IL13, IL-15, IL-21, IL-23, IL-27, TNF or their variant/mimic. In some embodiment, the cytokine is an interferon, IL-2, IL-7, TNF or their variant/mimic. In some embodiments, the compound to be tested using the method disclosed herein is a cytokine variant/mimic that is at least 10%, 20%, 30%, 40%, 50%, 60%, 70%, 80%, 90% or 99% identical to a naturally occurring cytokine. For example, the protein tested using the method disclosed herein can be at least 50% or 90% identical to a known cytokine in sequence. In some embodiments, the one or more proteins differ from a known protein by at least one amino acid, two amino acids, three amino acids, four amino acids, or five amino acids. For example, the cytokine mimics can differ from a known or naturally occurring cytokine by at least one amino acid, two amino acids, three amino acids, four amino acids, or five amino acids. The variation can be substitution, deletion or insertion of at least one residue(s).

In some embodiments, the number of proteins tested using the method disclosed herein varies. In some examples, the one or more proteins can comprise at least 100, 1000 or 10000 proteins.

In some embodiments, the target is an antibody or a cytokine receptor. In some embodiment, the target is a cytokine receptor, such as an interferon receptor, interleukin-10 receptor, interleukin-20 receptor, interleukin-22 receptor, IL-2 receptor, IL-3 receptor, IL-4 receptor, IL-5 receptor, IL-6 receptor, IL-7 receptor, IL-9 receptor, IL-11 receptor, IL-12 receptor, IL13 receptor, IL-15 receptor, IL-21 receptor, IL-23 receptor, IL-27 receptor, TNF receptor or their variant. In some embodiment, the target is an interferon receptor, IL-2 receptor, IL-7 receptor, TNF receptor or their variant. In some embodiments, the target is a complex, a subunit or a fragment of a cytokine receptor. In some embodiment, the target is a subunit of a cytokine receptor. In some embodiment, the target is IL-2Rβ, IL-2Rγc, IL-7Rαn or TNFR1. In some embodiments, the target is a cytokine receptor variant that is at least 10%, 20%, 30%, 40%, 50%, 60%, 70%, 80%, 90% or 99% identical to a naturally occurring cytokine receptor or a complex, a subunit or a fragment of a naturally occurring cytokine receptor. The variation can be substitution, deletion or insertion of at least one residue(s).

In some embodiments, the target is a cytokine receptor, and the compounds to be tested for binding strength with the target are a library of variants of a known binding partner of that target. The variants can bind with the target with greater or less binding strength compared to the known binding partner of that target. The variant can also be a non-binder. For example, the target can be IL-2Rβ, the known binding partner is Neo-2/15 and the compounds are variants of Neo-2/15. The library of variants can be a site saturation library, in which each variant differs from Neo-2/15 by one residue and the library comprises all possible substitutions at each position along the length of Neo-2/15.

### Protein Display

In some embodiments, the compound to be tested in binding assay disclosed herein is a protein, a peptide, or a fragment thereof. The protein or peptide can be displayed by a display library (e.g., phage display library, ribosome display library, mRNA display library, and yeast display library). The display library can be a cell surface display library (e.g., yeast display library), a display library utilizing no cells (e.g., phage display library, ribosome display library, and mRNA display library) or a synthetic library.

Cell surface display library can express a protein or peptide on the surface of prokaryotic (e.g., bacteria) or eukaryotic cells (e.g., bacteria, yeast, insect, and mammalian cells). For example, the peptide display cell can be a cell type commonly used for protein expression, such as yeast cell, a mammalian cell (e.g., Chinese hamster ovary (CHO) cells, baby hamster kidney (BHK) cells, NS0 myeloma and Sp2/0 hybridoma mouse cell lines, human embryonic kidney cells 293 (HEK293) and HT-1080 human cells), an insect cell or a plant cell. Examples of mammalian cell can be used for displaying of peptide include human cell lines used or developed for biopharmaceutical protein production (e.g., HEK293, PER.C6, CEVEC's amniocyte production (CAP), AGE1.HN, HKB-11 and HT-1080 cells), several derivatives of HEK293 cells (e.g., HEK293-T and HEK293-EBNA1), non-human mammalian expression systems (e.g., BHK-21 cells, murine NSO myeloma and Sp2/0 hybridoma cells), and “difficult to transfect” cells (e.g., T cells).

The protein or peptide can, for example, be coupled to a protein present at a cell surface and, by association with the cellular protein, can be displayed at the surface of the cell. Typically, the genetic information encoding the peptide or protein for display can be introduced into the cell (e.g., bacteria, yeast, insect, or mammalian cell) in the form of a polynucleotide element, such as a plasmid, using known techniques in the field of art. Any suitable delivery method can be used for introducing a polynucleotide element, e.g., plasmid, into a cell. Non-limiting examples of methods introducing the genetic information encoding the peptide or protein for display include, for example, viral or bacteriophage infection, transfection, conjugation, protoplast fusion, lipofection, electroporation, calcium phosphate precipitation polyethyleneimine (PEI)-mediated transfection, DEAE-dextran mediated transfection, liposome-mediated transfection, particle gun technology, calcium phosphate precipitation, direct microinjection, use of cell permeable peptides, and nano-particle mediated nucleic acid delivery. Conventional viral and non-viral based gene transfer methods can be used. Non-viral vector delivery systems can include DNA plasmids, RNA, naked nucleic acid, and nucleic acid complexed with a delivery vehicle, such as a liposome. Viral vector delivery systems can include DNA and RNA viruses, which can have either episomal or integrated genomes after delivery to the cell. Methods of non-viral delivery of nucleic acids can include lipofection, nucleofection, microinjection, biolistics, virosomes, liposomes, immunoliposomes, polycation or lipid-nucleic acid conjugates, naked DNA, artificial virions, and agent-enhanced uptake of DNA. Cationic and neutral lipids that are suitable for efficient receptor-recognition lipofection of polynucleotides can be used. The preparation of lipid-nucleic acid complexes, including targeted liposomes such as immunolipid complexes, can be used. In some cases, expressing the peptide or protein comprises editing a cell genome via an integrase, recombinase, or Cas protein.

The cell can use the exogenous genetic information to produce the protein or peptide to be displayed. The genetic information (e.g., sequence-based information) can later be interrogated, for example by sequencing analysis, to determine the identity of a protein or peptide (e.g., sequence of the nucleic acid sequence encoding the protein or peptide) identified by sequencing. The identity of the protein or peptide can also be determined by interrogating the sequence information of a nucleic tag added to the nucleic acid sequence encoding the protein or peptide, before introducing the nucleic acid sequence encoding the protein or peptide into the cell.

In some cases, a library of cell-surface displayed proteins (e.g., yeast displayed) generated herein can be subjected to binding or interaction assays to determine the affinity between the displayed protein and a target. The library can include a plurality of proteins or peptides having different amino acid sequences displayed on a cell surface. Each member of the library can have unique properties, binding a target with affinity that is different from or the same as that of another member of the library.

In some cases, the protein or peptide is displayed without using cells. In some embodiments, the vector displaying the protein or peptide is a virus. In some embodiments, the vector is a liposome or an exosome. Non-limiting examples of technologies that do not utilize cells include phage display, mRNA display, and ribosome display. A protein or peptide of interest can be displayed, for example, on a phage by inserting the protein coding sequence into a phage genome (e.g. in a phage coat protein gene). When the phage DNA is expressed as phage proteins, it can display the protein of interest on the surface of the phage, and package the corresponding DNA inside the phage capsid. In some cases, the protein coding sequence can be linked to a tag sequence which can be used to identify the protein of interest.

In an exemplary embodiment of the present invention, the display package is a phage particle which comprises a peptide domain fusion coat protein that includes the amino acid sequence of a test peptide domain. Thus, a library of replicable phage vectors, especially phagemids, encoding a library of peptide domain fusion coat proteins is generated and used to transform suitable host cells. In a preferred embodiment, each individual phage particle of the library includes a copy of the corresponding phagemid encoding the peptide domain fusion coat protein displayed on the surface of that package. Exemplary phage for generating the present variegated peptide domain libraries include M13, f1, fd, If1, Ike, Xf, Pf1, Pf3, λ, T2, T3, T4, T7, P2, P4, Phi X-174, MS2, Bacillus phage Phi29 and f2.

In addition to commercially available kits for generating phage display libraries (e.g. the Pharmacia Recombinant Phage Antibody System, catalog no. 27-9400-01; and the Stratagene SurβAP™ phage display kit, catalog no. 240612), examples of methods and reagents particularly amenable for use in generating the variegated peptide domain display library of the present invention can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Marldand et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) RN4S 88:7978-7982. These systems can, with modifications described herein, be adapted for use in the subject method.

In some cases, a library of phage displayed proteins generated herein can be subjected to binding or interaction assays to determine the binding affinity of each member of the library with a target. In some cases, a library of phage displayed proteins generated herein can be subjected to binding or interaction assays to determine the affinity between the displayed protein and a target. The library can include a plurality of proteins or peptides having different amino acid sequences displayed on a cell surface. Each member of the library can have unique properties, binding a target with affinity that is different from or the same as that of another member of the library.

In some embodiments, a protein of interest is produced by mRNA display. In mRNA display, a translated protein can be associated with its coding mRNA via a linkage (e.g., a puromycin linkage). In some cases, the coding mRNA can be linked to a tag sequence which can be used to identify the protein of interest. In some cases, a library of mRNA displayed proteins generated herein can be subjected to binding or interaction assays to determine the affinity between the displayed protein and a target. The library can include a plurality of proteins or peptides having different amino acid sequences displayed on a cell surface. Each member of the library can have unique properties, binding a target with affinity that is different from or the same as that of another member of the library.

In some embodiments, a protein of interest is produced by ribosome display. In ribosome display, the translated protein can be associated with its coding mRNA and a ribosome. In some cases, the coding mRNA may be linked to a tag sequence which can be used to identify the protein of interest. In some cases, a library of ribosome displayed proteins generated herein can be subjected to binding or interaction assays to determine the affinity between the displayed protein and a target. The library can include a plurality of proteins or peptides having different amino acid sequences displayed on a cell surface. Each member of the library can have unique properties, binding a target with affinity that is different from or the same as that of another member of the library.

The library can display random proteins or peptides (random display library) or a few proteins or peptides and/or mutants derived from these few proteins or peptides (targeted display library). In some examples, the library can display fragments of the proteins or peptides instead of the full-size proteins or peptides. The display library can display at least one protein or peptide comprising at least one mutation. In some embodiments, the size of the displayed protein or peptide is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1 It may include, but is not limited to, 000, about 1100, about 1200, about 1300, about 1400, about 1500, about 1750, about 2000, about 2250, about 2500 amino acid residues or more.

In some embodiments, the size of the library varies. For example, the display library can comprise at least 100, 200, 300, 400, 500, 600, 700, 800, 900, 1000, 2000, 3000, 4000, 5000, 6000, 7000, 8000, 9000, 10000, 20000, 30000, 40000, 50000, or a number between any two of these values. In some embodiments, the proteins displayed on one vector are the same. In some embodiments, the proteins displayed on one vector are different. In some embodiment, the proteins displayed on at least two of the vectors are different. In some embodiments, the proteins displayed on at least five, ten, fifty, a hundred, a thousand, or ten thousand of the plurality of vectors are different. At least one of the displayed proteins can differ from one or more of the remaining displayed proteins in the library by at least 1 amino acid (e.g., 1 amino acids, 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, 15 amino acids, 20 amino acids, 25 amino acids, 30 amino acids, 35 amino acids, 40 amino acids, 45 amino acids, 50 amino acids, or a number between any two of these values). In some embodiments, at least one of the encoded proteins differ from each of the remaining encoded proteins by at least 1 amino acid (e.g., 1 amino acids, 2 amino acids, 3 amino acids, 4 amino acids, 5 amino acids, 6 amino acids, 7 amino acids, 8 amino acids, 9 amino acids, 10 amino acids, 11 amino acids, 12 amino acids, 13 amino acids, 14 amino acids, 15 amino acids, 20 amino acids, 25 amino acids, 30 amino acids, 35 amino acids, 40 amino acids, 45 amino acids, 50 amino acids, or a number between any two of these values).

### The Binding Step

A method for using displayed proteins in a binding or interaction assay can comprise one or more of the following operations. A plurality copies of a target is mixed with a protein or peptide display library (e.g., phage display library, ribosome display library, mRNA display library, yeast display library, synthetic library, etc.) and incubated to allow for the target and displayed proteins to bind. In some cases, the target is a cytokine receptor (e.g., IL-2Rβ, IL-2Rγc, IL-7Rα, and TNFR1) and the cytokine receptor binds to a displayed protein (e.g., an IL-2 mimic). Mixing and binding the plurality copies of the target with the protein display library can comprise inoculating the plurality of copies of the target with the protein display library under conditions that allow the target to bind with displayed proteins of the protein display library. The target can bind to a folded or unfolded polypeptide. For example, to allow specific binding between the target and the displayed protein or peptide, the target and the protein display library can be mixed and incubated under conditions suitable for the target to bind with the protein display library. The incubation can be conducted at a temperature ranging from 25° C. to 45° C. (e.g., at 25° C., 30° C., 35° C., 40° C., 45° C. or a range between any two of these values). The incubation can be conducted for a period of time ranging from 5 min to 24 h (e.g., for 5 min, 10 min, 15 min, 20 min, 25 min, 30 min, 35 min, 40 min, 45 min, 50 min, 55 min, 1 h, 2 h, 3 h, 5 h, 6 h, 7 h, 8 h, 9 h, 10 h, 11 h, 12 h, 15 h, 18 h, 21 h, 24 h or a range between any two of these values).

In some embodiments, the target is immobilized or attached to a bead, or the target is partially or entirely embedded in a bead. In some embodiments, the plurality copies of the targets are reversibly attached to, covalently attached to, or irreversibly attached to the bead. In some embodiments, the target is covalently conjugated to the bead. In some embodiments, one or more additional targets are immobilized or attached to the bead, or one or more additional targets are embedded in the bead. The number of the additional targets can be about, at least, or at most 1, 2, 3, 4, 5, 6, 7, 8, 9, 10, 20, 30, 40, 50, 60, 70, 80, 90 or 100.

In some embodiments, the bead is a solid bead or a semi-solid bead. In some embodiments, the bead is a hydrogel bead, a polymer bead, or a magnetic bead. In some embodiments, the bead is a gel bead. The gel bead can be degradable upon application of a stimulus. The stimulus can comprise a thermal stimulus, a chemical stimulus, a biological stimulus, a photo-stimulus, or a combination thereof. The bead can be a magnetic bead. In some embodiments, the plurality copies of the targets are immobilized on a surface of the beads.

A size or dimension (e.g., length, width, depth, radius, or diameter) of a bead can be different in different embodiments. In some embodiments, a size or dimension of one, or each, bead is, is about, is at least, is at least about, is at most, or is at most about, 1 nanometer (nm), 2 nm, 3 nm, 4 nm, 5 nm, 6 nm, 7 nm, 8 nm, 9 nm, 10 nm, 11 nm, 12 nm, 13 nm, 14 nm, 15 nm, 16 nm, 17 nm, 18 nm, 19 nm, 20 nm, 21 nm, 22 nm, 23 nm, 24 nm, 25 nm, 26 nm, 27 nm, 28 nm, 29 nm, 30 nm, 31 nm, 32 nm, 33 nm, 34 nm, 35 nm, 36 nm, 37 nm, 38 nm, 39 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 56 nm, 57 nm, 58 nm, 59 nm, 60 nm, 61 nm, 62 nm, 63 nm, 64 nm, 65 nm, 66 nm, 67 nm, 68 nm, 69 nm, 70 nm, 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm, 90 nm, 91 nm, 92 nm, 93 nm, 94 nm, 95 nm, 96 nm, 97 nm, 98 nm, 99 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 310 nm, 320 nm, 330 nm, 340 nm, 350 nm, 360 nm, 370 nm, 380 nm, 390 nm, 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, 750 nm, 760 nm, 770 nm, 780 nm, 790 nm, 800 nm, 810 nm, 820 nm, 830 nm, 840 nm, 850 nm, 860 nm, 870 nm, 880 nm, 890 nm, 900 nm, 910 nm, 920 nm, 930 nm, 940 nm, 950 nm, 960 nm, 970 nm, 980 nm, 990 nm, 1000 nm, 2 micrometer (um), 3 μm, 4 μm, 5 μm, 6 um, 7 μm, 8 μm, 9 μm, 10 μm, 20 μm, 30 μm, 40 μm, 50 μm, 60 μm, 70 μm, 80 μm, 90 μm, 100 um, 110 μm, 120 μm, 130 μm, 140 μm, 150 μm, 160 μm, 170 μm, 180 μm, 190 μm, 200 μm, 210 um, 220 μm, 230 μm, 240 μm, 250 μm, 260 μm, 270 μm, 280 μm, 290 μm, 300 μm, 310 μm, 320 um, 330 μm, 340 μm, 350 μm, 360 μm, 370 μm, 380 μm, 390 μm, 400 μm, 410 μm, 420 μm, 430 um, 440 μm, 450 μm, 460 μm, 470 μm, 480 μm, 490 μm, 500 μm, or a number or a range between any two of these values. For example, a size or dimension of one, or each, bead is about 1 nm to about 100 μm.

The volume of one, or each, bead can be different in different embodiments. The volume of one, or each, bead can be, be about, be at least, be at least about, be at most, or be at most about, 1 nm³, 2 nm³, 3 nm³, 4 nm³, 5 nm³, 6 nm³, 7 nm³, 8 nm³, 9 nm³, 10 nm³, 20 nm³, 30 nm³, 40 nm3, 50 nm³, 60 nm³, 70 nm³, 80 nm³, 90 nm³, 100 nm³, 200 nm³, 300 nm³, 400 nm³, 500 nm³, 600 nm3, 700 nm³, 800 nm³, 900 um3, 1000 nm³, 10000 nm³, 100000 μm³, 1000000 nm³, 10000000 nm3, 100000000 μm³, 1000000000 nm³, 2 μm³, 3 μm³, 4 μm³, 5 μm³, 6 μm³, 7 μm³, 8 μm³, 9 μm³, 10 μm³, 20 μm³, 30 μm³, 40 μm³, 50 μm³, 60 μm³, 70 μm³, 80 μm³, 90 μm³, 100 μm³, 200 μm³, 300 μm³, 400 μm³, 500 μm³, 600 μm³, 700 μm³, 800 μm³, 900 μm³, 1000 μm³, 10000 μm³, 100000 um3, 1000000 μm³, or a number or a range between any two of these values. The volume of one, or each, bead can be, be about, be at least, be at least about, be at most, or be at most about, 1 nanoliter (nl), 2 nl, 3 nl, 4 nl, 5 nl, 6 nl, 7 nl, 8 nl, 9 nl, 10 nl, 11 nl, 12 nl, 13 nl, 14 nl, 15 nl, 16 nl, 17 nl, 18 nl, 19 nl, 20 nl, 21 nl, 22 nl, 23 nl, 24 nl, 25 nl, 26 nl, 27 nl, 28 nl, 29 nl, 30 nl, 31 nl, 32 nl, 33 nl, 34 nl, 35 nl, 36 nl, 37 nl, 38 nl, 39 nl, 40 nl, 41 nl, 42 nl, 43 nl, 44 nl, 45 nl, 46 nl, 47 nl, 48 nl, 49 nl, 50 nl, 51 nl, 52 nl, 53 nl, 54 nl, 55 nl, 56 nl, 57 nl, 58 nl, 59 nl, 60 nl, 61 nl, 62 nl, 63 nl, 64 nl, 65 nl, 66 nl, 67 nl, 68 nl, 69 nl, 70 nl, 71 nl, 72 nl, 73 nl, 74 nl, 75 nl, 76 nl, 77 nl, 78 nl, 79 nl, 80 nl, 81 nl, 82 nl, 83 nl, 84 nl, 85 nl, 86 nl, 87 nl, 88 nl, 89 nl, 90 nl, 91 nl, 92 nl, 93 nl, 94 nl, 95 nl, 96 nl, 97 nl, 98 nl, 99 nl, 100 nl, or a number or a range between any two of these values. For example, the volume of one, or each, bead is about 1 nm3 to about 1000000 um³.

The number of beads mixed with the protein display library can be different in different embodiments. In some embodiments, the number of beads is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number of beads can be at least 1000 beads.

In some embodiments, the plurality of targets attached to each bead are the same. In some embodiments, different targets are attached to the same beads. In some embodiments, the plurality of targets attached to each bead of a plurality of beads that are mixed with one protein display library are the same. In some embodiments, targets attached to different beads that are mixed with one protein display library are different. In some embodiments, targets attached to all beads that are mixed with one protein display library are the same.

Following the incubation, the vectors that are bound to the target can be separated from the vectors that are not bound to the target. In some embodiments, separating the vectors that are bound to the target from the vectors that are not bound to the target comprises removing the vectors displaying a protein not bound to the target.

### Barcoding

In some embodiments, barcoding the nucleic acids from the vectors that are bound to the target comprises barcoding the nucleic acids from the vectors that are bound to the target with a plurality of barcode oligonucleotides. Each barcode oligonucleotide of the plurality of barcode oligonucleotides can comprise a barcoding primer and a barcode sequence. In some embodiments, the nucleic acid in each of the plurality of vectors comprises a barcoding primer binding region capable of binding to the barcoding primer. In some embodiments, the barcode sequence comprises a randomer sequence. In some embodiments, the randomer sequence is a unique molecular identifier (UMI) sequence. In some embodiments, each barcode oligonucleotide of the plurality of barcode oligonucleotides can further comprise a PCR primer binding region/sequence and/or one or more sequencing primer sequences. In some embodiments, barcoding the nucleic acids from the vectors of the display library comprises hybridizing the barcoding primer with the barcoding primer binding region. The vectors can be those bound to the target after a binding assay or those taken before a binding assay. The method disclosed herein can further comprise obtaining the nucleic acid encoding a protein before barcoding. For example, the method can comprise obtaining genomic nucleic acid from the phage, which comprises the nucleic acid encoding the protein.

### Unique Molecular Identifier (UMI)

The number (or percentage) of UMIs of barcode oligonucleotides added to each binding assay with different sequences can be different in different embodiments. In some embodiments, the number of UMIs of barcode oligonucleotides added to each binding assay with different sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value. In some embodiments, the percentage of UMIs of barcode oligonucleotides added to each binding assay with different sequences is, is about, is at least, is at least about, is at most, or is at most about, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or a number or a range between any two of these values. In some embodiments, the sequence of the UMI in each barcode oligonucleotide of the plurality of barcode oligonucleotides is different from the sequences of the UMIs in any other barcode oligonucleotides of the plurality of barcode oligonucleotides.

The number of barcode oligonucleotides added to each binding assay with UMIs having a particular sequence (or an identical sequence) can be different in different embodiments. In some embodiments, the number of barcode oligonucleotides added to each binding assay with UMIs having a particular sequence (or an identical sequence) is, is about, is at least, is at least about, is at most, or is at most about, 1, 2, 3, 4, 5, 6, 7, 8, 9, 10, 20, 30, 40, 50, 60, 70, 80, 90, 100, or a number or a range between any two of these values. For example, the UMIs of two barcode oligonucleotides added to each binding assay can comprise a particular sequence (or an identical sequence).

The length of a UMI of a barcode oligonucleotide (or each UMI of a plurality of barcode oligonucleotides or all UMIs of the plurality of barcode oligonucleotides) can be different in different embodiments. In some embodiments, a UMI of a barcode oligonucleotide (or each UMI of a plurality of barcode oligonucleotides or all UMIs of the plurality of barcode oligonucleotides) is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length. For example, a UMI can be at least 15 nucleotides in length.

The number of unique UMI sequences can be different in different embodiments. In some embodiments, the number of unique UMI sequences is, is about, is at least, is at least about, is at most, or is at most about, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these value.

### Barcoding Primer

The barcoding primer can hybridize with a nucleotide sequence (barcoding primer binding region) of the display library comprising the sequence encoding the displayed protein. The barcoding primer binding region can be upstream of or within the sequence encoding the displayed protein. In some embodiments, the barcoding primer binding region is 1-500 bp (e.g., 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp or a number between any two of these values) upstream of the sequence encoding the displayed protein. For example, the barcoding primer binding region can be 100-300 bp upstream of the sequence encoding the displayed protein. In some embodiments, the barcoding primer binding region is, is about, is at least, is at least about, is at most, or is at most about, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number between any two of these values, in length.

In some embodiments, the barcoding primer is, is about, is at least, is at least about, is at most, or is at most about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or a number or a range between any two of these values, nucleotides in length.

### Synthesis of Single-Stranded Barcoded Nucleic Acids

In some embodiments, barcoding the nucleic acids from the vectors comprises generating a single-stranded DNA comprising the barcode sequence, using the barcoding primer and the nucleic acid encoding a protein as template. In some embodiments, barcoding from the vectors can comprise extending the plurality of barcode oligonucleotides using the nucleic acids encoding displayed proteins as templates to generate partially single-stranded/partially double-stranded barcoded nucleic acids hybridized to the nucleic acids encoding displayed proteins. The partially single-stranded/partially double-stranded nucleic acids hybridized to the nucleic acids encoding displayed proteins can be separated by denaturation (e.g., heat denaturation or chemical denaturation using for example, sodium hydroxide) to generate single-stranded barcoded nucleic acids of the nucleic acids encoding displayed proteins. The single-stranded barcoded nucleic acids can comprise a barcode oligonucleotide and an oligonucleotide complementary to the nucleic acids encoding displayed proteins. In some embodiments, the single-stranded barcoded nucleic acids can be generated by reverse transcription using a reverse transcriptase. For example, the single-stranded barcoded nucleic acids can be generated by using a DNA polymerase.

In some embodiments, the single-stranded barcoded nucleic acids can be DNA produced by extending a barcode oligonucleotide using a DNA (e.g., dsDNA) encoding a displayed protein as a template. For example, a DNA polymerase can be used to generate a DNA by extending a barcode oligonucleotide hybridized to a DNA. In some embodiments, the barcoded nucleic acid from each of the vectors comprises the coding sequence of the protein and a barcode sequence selected from a diverse set of barcode sequences. In some embodiments, the barcode sequence comprises a randomer sequence. In some embodiments, the randomer sequence is a unique molecular identifier (UMI) sequence. In some embodiments, the single-stranded DNA is generated through a single cycle of PCR.

In some embodiments, the single-stranded barcoded nucleic acids can be cDNA produced by extending a barcode oligonucleotide using an RNA (e.g., mRNA) encoding a displayed protein as a template. The single-stranded barcoded nucleic acids can be further extended using a template switching oligonucleotide (TSO). A TSO is an oligo that hybridizes to untemplated C nucleotides added by a reverse transcriptase during reverse transcription. The TSO can be introduced into the assay together with the reverse transcription reagents. For example, a reverse transcriptase can be used to generate a cDNA by extending a barcode oligonucleotide hybridized to an RNA. After extending the barcode oligonucleotide to the 5′-end of the RNA, the reverse transcriptase can add one or more nucleotides with cytosine (C) bases (e.g. two or three) to the 3′-end of the cDNA. The TSO can include one or more nucleotides with guanine (G) bases (e.g. two or more) on the 3′-end of the TSO. The nucleotides with G bases can be ribonucleotides. The G bases at the 3′-end of the TSO can hybridize to the cytosine bases at the 3′-end of the cDNA. The reverse transcriptase can further extend the cDNA using the TSO as the template to generate a cDNA with the reverse complement of the TSO sequence on its 3′-end. The barcoded nucleic acid can include the barcode sequences (e.g., UMI) on the 5′-end and a TSO sequence at its 3′-end.

The single-stranded barcoded nucleic acids can be separated from the template nucleic acids encoding displayed proteins by digesting the template sample nucleic acids (e.g., using RNase), by chemical treatment (e.g., using sodium hydroxide), by hydrolyzing the template nucleic acids encoding displayed proteins, or via a denaturation or melting process by increasing the temperature, adding organic solvents, or increasing pH. Following the melting process, the nucleic acids encoding displayed proteins can be removed (e.g. washed away).

### PCR Primer-Binding Region

In some embodiments, each barcode oligonucleotide of the plurality of barcode oligonucleotides comprises a first polymerase chain reaction (PCR) primer-binding region. The first PCR primer-binding region can comprise a Read 1 sequence.

### Barcoded Nucleic Acid Amplification

In some embodiments, the method comprises amplifying the barcoded nucleic acids to generate amplified barcoded nucleic acids. Amplifying the barcoded nucleic acids can comprise amplifying the barcoded nucleic acids using polymerase chain reaction (PCR) to generate the amplified barcoded nucleic acids. For example, the barcode oligonucleotide can include a first polymerase chain reaction (PCR) primer-binding sequence (e.g., a Read 1 sequence). The first PCR primer-binding sequence can be used to amplify the barcoded nucleic acid. For example, the barcode oligonucleotide can include a first polymerase chain reaction (PCR) primer-binding sequence (e.g., a Read 1 sequence). A first primer comprising the sequence of first PCR primer-binding sequence and a second primer comprising a random sequence (e.g., a random hexamer) can be used to amplify the barcoded nucleic acid. The second primer can include one or more non-random sequences. For example, the second primer can hybridize to a region downstream of or within the coding sequence of the displayed protein. In some embodiments, the second primer can hybridize to a region 1-500 bp (e.g., 1 bp, 2 bp, 3 bp, 4 bp, 5 bp, 6 bp, 7 bp, 8 bp, 9 bp, 10 bp, 20 bp, 30 bp, 40 bp, 50 bp, 60 bp, 70 bp, 80 bp, 90 bp, 100 bp, 200 bp, 300 bp, 400 bp, 500 bp or a number between any two of these values) downstream of the coding sequence of the displayed protein.

For example, analyzing the nucleic acids encoding a protein in the protein display library can comprise amplifying the barcoded nucleic acid to generate amplified barcoded nucleic acids; processing the amplified barcoded nucleic acids to generate processed barcoded nucleic acids; and sequencing the barcoded nucleic acids, or products thereof, to obtain sequencing information. Amplifying the barcoded nucleic acids can comprise amplifying the barcoded nucleic acids using polymerase chain reaction (PCR) to generate the amplified barcoded nucleic acids. Sequencing the barcoded nucleic acids can comprise sequencing the processed barcoded nucleic acids.

### Enrichment

In some embodiments, the method comprises enriching the nucleic acid encoding a protein in the protein display library using one or more enrichment primers. Enriching the nucleic acid encoding a protein in the protein display library can comprise enriching the nucleic acid encoding a protein in the protein display library using primers specific to the nucleic acid encoding a protein in the protein display library when amplifying the barcoded nucleic acids. For example, a first primer comprising the sequence of first PCR primer-binding sequence and a second primer comprising a sequence specific to a nucleic acid encoding a protein in the protein display library (e.g., a partial sequence of the nucleic acid encoding a protein in the protein display library, or a reverse complement thereof) can be used to amplify the barcoded nucleic acid. The second primer can include additional one or more sequences, such as a second PCR primer-binding sequence (e.g., a Read 2 sequence). Enriching the nucleic acid targets can comprise enriching the nucleic acid targets using the enrichment primers of a panel. The panel can be a customizable panel.

### Sequencing Library Construction

In some embodiments, the method comprises processing barcoded nucleic acids to generate processed barcoded nucleic acids. For example, the method can include enzymatic fragmentation of the barcoded nucleic acids, end repair of fragmented nucleic acids, A-tailing of fragmented nucleic acids that have been end-repaired, and ligation of a double stranded adaptor with a second PCR primer-binding sequence (e.g., a Read 2 sequence). Sequencing the barcoded nucleic acids can comprise sequencing the processed barcoded nucleic acids.

In some embodiments, processing the amplified barcoded nucleic acids comprises fragmenting the amplified barcoded nucleic acids to generate fragmented barcoded nucleic acids. Fragmenting the amplified barcoded nucleic acids can comprise fragmenting the amplified barcoded nucleic acids enzymatically to generate the fragmented barcoded nucleic acids. Fragmented barcoded nucleic acids can undergo end-repair and A-tailing (to add a few nucleotides with adenosine (A) bases). Processing the amplified barcoded nucleic acids can comprise adding a second polymerase chain reaction (PCR) primer-binding sequence. The second PCR primer-binding sequence can comprise a Read 2 sequence. For example, a double-stranded adaptor comprising the second PCR primer-binding sequence can be ligated to the fragmented barcoded nucleic acids after, for example, end repair and A tailing using a ligase. The adaptor can include a few thymine (T) bases that can hybridize to the few A bases added by A tailing. Processing the amplified barcoded nucleic acids can comprise generating processed barcoded nucleic acids comprising sequencing primer sequences from the fragmented barcoded nucleic acids (e.g., after end repair, A tailing, and ligation of an adaptor comprising the second PCR primer-binding sequence) using PCR. The sequencing primer sequences can comprise a P5 sequence and a P7 sequence. For example, a pair of PCR primers can be sued to add the sequencing primer sequences. A first PCR primer can comprise a P5 sequence and a Read 1 sequence (from 5′-end to 3′-end. A second PCR primer can comprise a P7 sequence and a Read 2 sequence (from 5′-end to 3′-end). A second PCR primer can comprise a P7 sequence, and a Read 2 sequence (from 5′-end to 3′-end). The pair of PCR primers can be used to generate processed nucleic acids by PCR. The processed nucleic acids can include a P5 sequence, a Read 1 sequence, a UMI, a coding sequence of the displayed protein or a portion thereof, a Read 2 sequence, and/or a P7 sequence (e.g., from 5′-end to 3′-end). In some embodiments, sequencing the barcoded nucleic acids, or products thereof, comprises sequencing products of the barcoded nucleic acids. Products of the barcoded nucleic acids can include the processed nucleic acids.

For example, analyzing the barcoded nucleic acids can comprise analyzing the sequencing information. Analyzing the sequencing information can comprise sequencing the barcoded nucleic acids, or products thereof. Amplifying the barcoded nucleic acids can comprise amplifying the barcoded nucleic acids using polymerase chain reaction (PCR) to generate the amplified barcoded nucleic acids. Sequencing the barcoded nucleic acids comprises sequencing amplified barcoded nucleic acids. Sequencing the barcoded nucleic acids comprises sequencing products of the barcoded nucleic acids each comprising a P5 sequence, a Read 1 sequence, a unique molecular identifier (UMI), a coding sequence of the displayed protein or a portion thereof, a Read 2 sequence, and/or a P7 sequence to obtain sequencing information.

### ANALYSIS

### Sequencing Barcoded Nucleic Acids

The method disclosed herein can comprise sequencing the plurality of barcoded nucleic acids or products thereof to obtain nucleic acid sequences of the plurality of barcoded nucleic acids. As used herein, a “sequence” can refer to the sequence, a complementary sequence thereof (e.g., a reverse, a compliment, or a reverse complement), the full-length sequence, a subsequence, or a combination thereof. The nucleic acids sequences of the barcoded nucleic acids can each comprise a sequence of a barcode oligonucleotide (e.g. the UMI) and a sequence of a nucleic acid encoding a displayed protein or a reverse complement thereof. In some embodiments, analyzing the barcoded nucleic acids comprises obtaining sequence information of the barcoded nucleic acids. In some embodiments, analyzing the barcoded nucleic acids comprises sequencing the amplified barcoded nucleic acids.

Barcoded nucleic acids can be sequenced using any suitable sequencing method identifiable to a person skilled in the art. For example, sequencing the barcoded nucleic acids can be performed using high-throughput sequencing, pyrosequencing, sequencing-by-synthesis, single-molecule sequencing, nanopore sequencing, sequencing-by-ligation, sequencing-by-hybridization, next generation sequencing, massively-parallel sequencing, primer walking, and any other sequencing methods known in the art and suitable for sequencing the barcoded nucleic acids generated using the methods herein described.

### Post-Sequencing Analysis and Determining Binding Affinity

The obtained nucleic acid sequences of the barcoded nucleic acids can be subjected to any downstream post-sequencing data analysis as will be understood by a person skilled in the art. The sequence data can undergo a quality control process to remove adapter sequences, low-quality reads, uncalled bases, and/or to filter out contaminants. The high-quality data obtained from the quality control can be mapped or aligned to a reference genome or assembled de novo.

In some embodiments, analyzing the barcoded nucleic acids comprises determining abundance of the barcoded nucleic acids generated from the vectors that are bound to the target.

In some embodiments, obtaining abundance of the barcoded nucleic acids comprises sequencing the barcoded nucleic acids and determining relative or absolute amount of sequence reads in sequences of the barcoded nucleic acids. In some embodiments, determining relative or absolute amount of sequence reads comprises determine the number of UMIs with different sequences associated with each coding sequence of the protein.

Analyzing the barcoded nucleic acids can comprise determining the number of UMIs with different sequences associated the nucleic acid encoding the same displayed protein. The number of a displayed protein bound to a target can be estimated using the number of UMIs with different sequences associated the nucleic acid encoding the same displayed protein. For example, the binding strength of a displayed protein with a target can be calculated using the number of UMIs with different sequences associated the nucleic acid encoding the same displayed protein. In some examples, the binding strength of a displayed protein with a target can be estimated using simulation. The simulation can calculate the most probable binding strength (e.g., dissociation constant) based on the number of UMIs with different sequences associated the nucleic acid encoding the same displayed protein obtained before and after a binding assay disclosed herein.

Determining the binding between the proteins encoded by the plurality of vectors and the target can comprise determining the binding affinity between the proteins encoded by the plurality of vectors and the target. The binding affinity can be reflected by any parameters disclosed herein (e.g., dissociation constant or apparent dissociation constant). In some embodiments, determining the binding between the proteins encoded by the plurality of vectors and the target comprises ranking the proteins by the binding affinity. In some embodiments, the method further comprises separating a portion of the plurality of vectors before contacting the plurality of vectors with the target. The portion of the plurality of vectors can be barcoded and analyzed using the method disclosed herein to obtain a pre-binding abundance of the nucleic acids. In some embodiments, determining the binding between the proteins encoded by the plurality of vectors and the target comprises comparing the pre-binding abundance of the nucleic acids and an abundance of the barcoded nucleic acids.

In some embodiments, the binding affinity obtained using the method disclosed herein is provided to a protein design model as an input. Inputing the binding affinity obtained using the method disclosed herein to a protein design model can improve the performance of the protein design model.

### Composition & Kit

Disclosed herein include compositions for determining the binding between one or more proteins and a target. In some embodiments, the composition for determining the binding strength between one or more proteins and the target comprises a plurality of beads and/or a plurality of barcode oligonucleotides of the present disclosure. The targets attached to each of the plurality of beads can be identical. The targets attached to all beads can be the same. The number of beads can be different in different embodiments. In some embodiments, the number of beads is, is about, is at least, is at least about, is at most, or is at most, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 200000, 300000, 400000, 500000, 600000, 700000, 800000, 900000, 1000000, 2000000, 3000000, 4000000, 5000000, 6000000, 7000000, 8000000, 9000000, 10000000, 20000000, 30000000, 40000000, 50000000, 60000000, 70000000, 80000000, 90000000, 100000000, 200000000, 300000000, 400000000, 500000000, 600000000, 700000000, 800000000, 900000000, 1000000000, or a number or a range between any two of these values. For example, the number beads can be at least 100 beads.

Disclosed herein include kits for determining the binding strength between one or more proteins and the target. In some embodiments, a kit for determining the binding strength between one or more proteins and the target comprises a composition comprising a plurality of beads and/or a plurality of barcode oligonucleotides of the present disclosure. The kit can comprise instructions of using the composition for determining the binding strength between each of the at least one protein and the target.

Disclosed herein includes methods of generating beads comprising the target. In some embodiments, a method of generating beads comprising the target comprises providing a plurality of beads each coated with a plurality copies of the same target. Disclosed herein includes methods of generating a plurality of barcode oligonucleotides of the present disclosure.

### EXAMPLES

Some aspects of the embodiments discussed above are disclosed in further detail in the following examples, which are not in any way intended to limit the scope of the present disclosure.

### Example 1 Quantitative, High Throughput Training Data Generation for Protein Mimetic Design With Protein CREATE

The goal of protein engineering is the ability to design a protein according to multiple bioengineer-determined specifications. Traditional human-guided workflows require both manual selection of candidate protein variants and multiple design-build-test cycles, while the information gained from one design task does not easily inform others. The combination of these factors makes protein design difficult, if not impossible, in many scenarios. The Protein CREATE overcame these constraints by applying an automated framework, in which candidate protein variants were selected by artificial intelligence algorithms that continuously learn from the data generated from experiments conducted in the loop.

To produce an experiment-in-the-loop protein design framework, the data collection pipeline was first optimized. To this end, the data collection pipeline began with a phage display and bead pulldown workflow. Then new methods were developed to optimize the assay for reduced bias and higher quantitative power. Phage acted as genetically encoded nanoparticles that enabled expression of barcoded protein libraries at scale. T7 bacteriophage was chosen as a display chassis due to its lower sequence bias than M13. Each assayed protein variant was encoded in the phage genome and expressed as a capsid fusion. Thus, on average, each phage contained 5-15 copies of protein variant fusion on its surface. By being displayed on the capsid surface of T7 Phage, the protein variants of interest can bind to beads or cells containing a target. Phage libraries were added to “bait,” e.g. beads carrying a target protein or cells. Then, bait was extracted and a subset of the phage genome was sequenced to identify bound phage.

The core of advance of the platform disclosed herein is the ability to append unique molecular identifiers (UMIs) to sequences encoding protein variants embedded within phage genomes, allowing the counting of individual molecules enriched in phage display experiments. In doing so, the UMIs allowed quantitative estimation of the binding association constant for large protein libraries using a novel computational Bayesian inference framework. The resulting quantitative binding measurement platform can determine dissociation constants at scale, relieving the critical bottleneck around generation of training data for machine learning based protein design models. Specifically, a primer containing a 15 bp randomized region bound to the region of the phage genome directly upstream of the variant sequence and served as a UMI to individually label phage-derived amplicons. The phages went through a single cycle of PCR using one primer comprising UMI to add the UMI to an amplified subset of the phage genomic DNA. This primer contained, from 5′ to 3′, Illumina P5 adapter sequence, Illumina read 1 primer, the UMI region (a 15 base random sequence barcode), and the phage annealing region. 15 bases were chosen as the UMI length, based on empirical data suggesting an optimal balance between being long enough to minimize the probability of overloading a barcode and short enough to maintain PCR efficiency. The single-stranded DNA product of the first cycle of PCR was then purified using solid-phase reverse immobilization (SPRI) beads and amplified in another round(s) of PCR. Multiple dilutions of the single stranded DNA product were made, amplified, and later sequenced in this second PCR step to minimize the risk of UMI saturation and add precision to the measurement.

A subset of the phage genome amplified in the above-mentioned PCR acted as a barcode that can be read via next generation sequencing (NGS). NGS allowed thousands to millions of phage variants, each carrying a different engineered protein, to be assayed in parallel. Sequencing these amplicons before and after a single bead pulldown enabled single-molecule estimation. Specifically, the primer that bound to the phage genome containing a UMI permitted identification of individual molecule counts from raw sequencing read data. Combining with simulations and a Bayesian inference framework, the apparent dissociation constants for each variant was estimated. To calculate the dissociation constant, the UMI was added to the phase display library before and after before and after bead binding. Addition and optimization of the UMI to phage-specific primers represented a novel use of the UMI outside its original context of single cell RNA sequencing. Processing of the phages before and after bead binding represents another novel development of the platform disclosed herein. Custom Python scripts have been developed for compatibility with the sequencing architecture disclosed herein and translate raw sequencing data into quantitative binding data for the variants investigated in that run.

Bayesian Inference Framework converted reads into K_{d} estimates. The post-sequencing analysis converted individual reads into individual molecules, assigned each molecule to a variant tested in the experiment, and then calculated how many more phages were observed after bead binding than expected. Estimates for dissociation constants were made using that difference combined with variant prevalence in the initial phage pool, which were used to calculate the total number of variants in the pool. The measurements were fed into a stochastic simulation of the binding experiment, in which the dissociation constant was estimated by maximizing the likelihood of observing the number of phages seen in the experiment post-bead binding.

To validate results, the development of the platform has focused on the detection of IL-2 receptor binders, given both intense interest in re-engineering IL-2 as a therapeutic target, and extensive characterization of its mutants. In particular, de novo IL-2 mimetics with improved binding kinetics, solubility, and thermostability have been designed. These neoleukin2/15 (neo2/15) variants were cloned and displayed using the phage platform disclosed.

Thus far, the platform disclosed herein has been able to track 15 variants in parallel and estimate the dissociation constants of 10 variants. It is important to note that the limits of the ability to scale the assay have not been approached for several reasons: the sequencer used for building the platform (e.g., Illumina Miseq) had only 1/1000 of the capacity of the most high- throughput sequencers (e.g., Illumina NovaSeq X). The number of variants assayed has also been limited to aid in troubleshooting the platform.

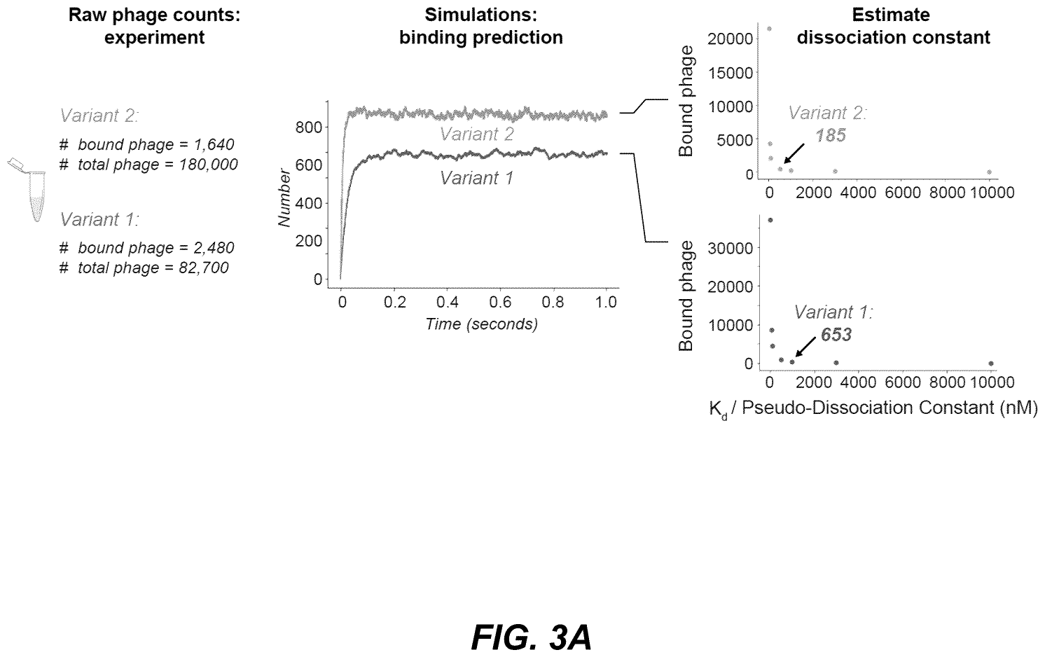

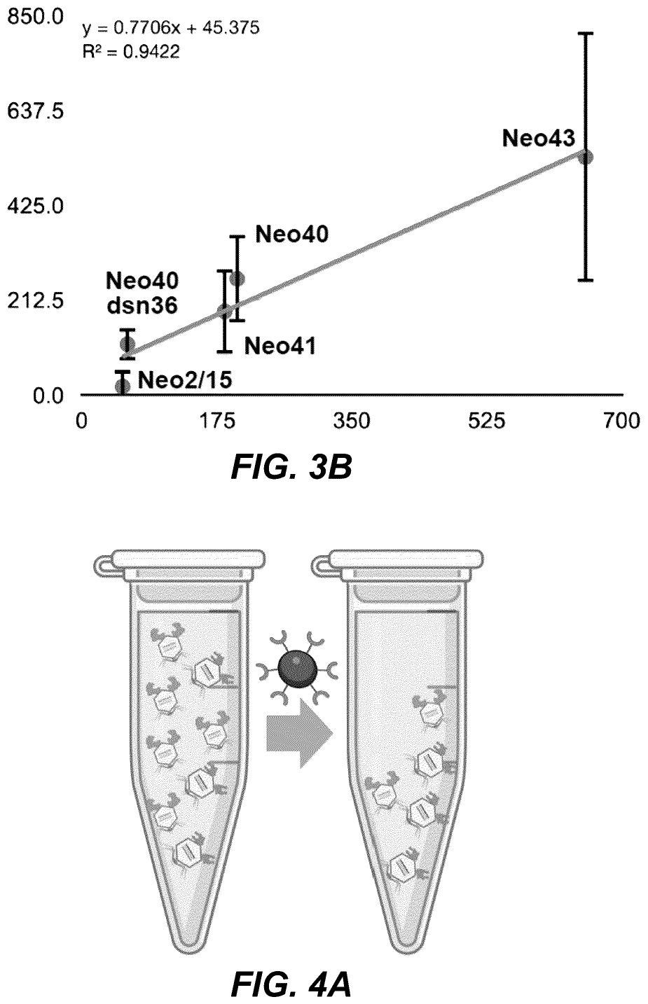

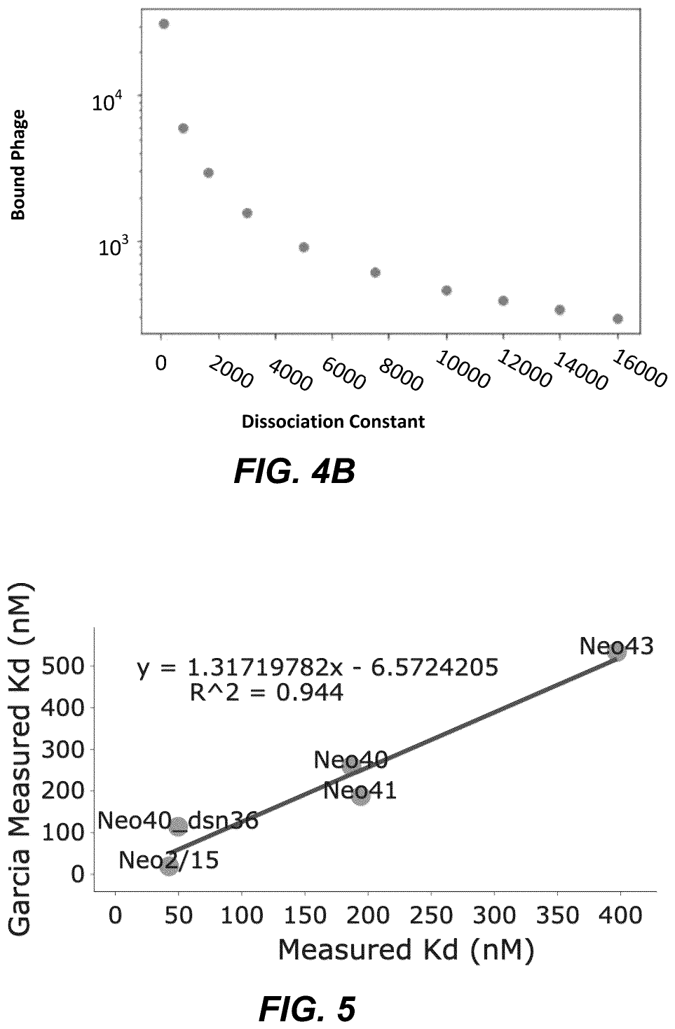

FIG. 5 shows a comparison of the variants tested with those having published dissociation constant values measured by surface plasmon resonance (SPR). SPR is a low-throughput but highly accurate measurement. The comparison shows close alignment with the predicted values, as all were within an order of magnitude of the true dissociation constant and, with the exception of one of the variants, have the correct rank order for binding strength. FIG. 3B also shows a comparison of dissociation constants measured using the platform disclosed herein to that measured using SPR. The measured values FIG. 3B are in Table 1 below.

TABLE 1
KD MEASUREMENTS OF NEOLEUKIN2/15
(NEO2/15) VARIANTS

| | Variant | | | | |
|---|---|---|---|---|---|
| | | | | | Neo40 |
| | Neo2/15 | Neo40 | Neo41 | Neo43 | Dsn36 |
| K_{d} 1 | 42.47 | 188.2 | 144.8 | 454 | 50.4 |
| K_{d} 2 | 21.06 | 109.5 | 117.1 | 4004 | 27.6 |
| K_{d} 3 | 94.18 | 305.6 | 293 | 851 | 98.6 |
| Mean K_{d} | 52.57 | 201.1 | 184.97 | 652.5 | 58.87 |
| Std Dev K_{d} | 37.59 | 98.68 | 94.58 | 280.72 | 36.25 |
| Garcia | 18.8 | 260 | 187 | 533.4 | 113.9 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “ a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “ a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method of determining binding between one or more proteins and a target comprising:
providing a plurality of vectors each comprises a nucleic acid encoding a protein, wherein each of the plurality of vectors displays the encoded protein on its surface and the encoded proteins from at least two of the plurality of vectors are different;
contacting the plurality of vectors with a target in a condition allowing the proteins displayed on the surfaces of the vectors to specifically bind the target;
separating the vectors that are bound to the target from the vectors that are not bound to the target;
barcoding the nucleic acids from the vectors that are bound to the target to generate barcoded nucleic acids;
analyzing the barcoded nucleic acids; and
determining the binding between the proteins encoded by the plurality of vectors and the target.

2. The method of claim 1, wherein determining the binding between the proteins encoded by the plurality of vectors and the target comprises determining the binding affinity between each of the encoded proteins and the target.

3. The method of claim 1, wherein separating the vectors that are bound to the target from the vectors that are not bound to the target comprises removing the vectors displaying a protein not bound to the target.

4. The method of claim 1, wherein the barcoded nucleic acid from each of the vectors comprises the coding sequence of the protein and a barcode sequence selected from a diverse set of barcode sequences.

5. The method of claim 1, wherein analyzing the barcoded nucleic acids comprises obtaining sequence information of the barcoded nucleic acids, and/or determining abundance of the barcoded nucleic acids generated from the vectors that are bound to the target.

6.-14. (canceled)

15. The method of claim 1, wherein the target is immobilized or attached to a bead, or the target is partially or entirely embedded in a bead.

16.-30. (canceled)

31. The method of claim 1, wherein the vector is a phage.

32. (canceled)

33. The method of claim 31, wherein the method comprises obtaining genomic nucleic acid from the phage, and wherein the phage genomic nucleic acid comprises the nucleic acid encoding the protein.

34.-38. (canceled)

39. The method of claim 1, wherein barcoding the nucleic acids from the vectors that are bound to the target comprises barcoding the nucleic acids from the vectors that are bound to the target with a plurality of barcode oligonucleotides.

40. The method of claim 39, wherein the barcode oligonucleotide comprises a barcode sequence and a barcoding primer, and wherein the nucleic acid in each of the plurality of vectors comprises a barcoding primer binding region capable of binding to the barcoding primer.

41. The method of claim 40, wherein the barcode sequence comprises a randomer sequence, wherein the randomer sequence is a unique molecular identifier (UMI) sequence, and wherein the length of the UMI is at least 6 bp, 10 bp, 15 bp, 20 bp, or 25 bp.

42. (canceled)

43. The method of claim 41, wherein the sequence of the UMI in each barcode oligonucleotide of the plurality of barcode oligonucleotides is different from the sequences of the UMIs in any other barcode oligonucleotides of the plurality of barcode oligonucleotides.

44. The method of claim 39, wherein the barcode oligonucleotide further comprises one or more PCR primer binding sites and/or one or more sequencing primer sequences.

45. The method of claim 40, wherein barcoding the nucleic acids from the vectors that are bound to the target comprises hybridizing the barcoding primer with the barcoding primer binding region.

46. The method of claim 40, wherein the barcoding primer binding region is about 1-300 bp upstream of the nucleic acid encoding a protein, and wherein the length of the barcoding primer binding region is about 5-100 bp.

47. (canceled)

48. The method of claim 40, wherein barcoding the nucleic acids from the vectors that are bound to the target comprises generating a single-stranded DNA comprising the barcode sequence, using the barcoding primer and the nucleic acid encoding a protein as template.

49. The method of claim 48, wherein the single-stranded DNA is generated through a single cycle of PCR.

50. The method of claim 1, further comprising separating a portion of the plurality of vectors before contacting the plurality of vectors with the target; and/or
barcoding and analyzing the portion of the plurality of vectors to obtain a pre-binding abundance of the nucleic acids,
wherein determining the binding between the proteins encoded by the plurality of vectors and the target comprises comparing the pre-binding abundance of the nucleic acids and an abundance of the barcoded nucleic acids.

51.-56. (canceled)

57. The method of claim 2, wherein the binding affinity is dissociation constant or apparent dissociation constant.

58.-62. (canceled)

63. A composition/kit comprising a bead and a plurality of barcode oligonucleotides of claim 39.
